(19) United States
(12) Patent Application Publication
(10) Pub. No.: US 2024/0381914 A1
(43) Pub. Date: Nov. 21, 2024

(54) METHOD FOR PREPARING A FOOD SOLID, FOOD SOLID COMPRISING KONJAC GLUCOMMANNAN AND USE THEREOF

(71) Applicant: Daniel SAMAIN, TOULOUSE (FR)

(72) Inventor: Daniel SAMAIN, TOULOUSE (FR)

(21) Appl. No.: 17/442,748

(22) PCT Filed: Mar. 13, 2020
(86) PCT No.: PCT/EP2020/056963

§ 371 Date: Apr. 28, 2022

(30) Foreign Application Priority Data
Mar. 26, 2019 (FR) 19 03114

(51) Int. Cl. A23L 19/10; A23P 30/25

(52) U.S. Cl. CPC A23L 19/115; A23P 30/25

(57) ABSTRACT

The invention relates to a process for preparing a food solid, in which is prepared a mixture:
of an amount of a flour comprising at least one polysaccharide, named heteromannan, chosen from the group formed from glucomannans and galactomannans, and
of an amount of an aqueous liquid composition, characterized in that said heteromannan is in an amount such that the ratio of the mass of said heteromannan in the mixture to the mass of said aqueous liquid composition in the mixture is between 5 % and 35%, and
   in that the mixture is prepared by vigorous stirring, via which a substantially homogeneous dispersion is formed, named pourable dispersion, of the flour in the aqueous liquid composition, and having a dynamic viscosity of less than 100 Pa·s, said pourable dispersion spontaneously changing to form an aqueous cohesive solid which is substantially free of free aqueous liquid composition and which has a dynamic viscosity greater than the dynamic viscosity of said pourable dispersion, and then
   a step of maturation of the aqueous cohesive solid and of hardening of the aqueous cohesive solid is performed so as to form the food solid, the formed food solid being non-adhesive and non-coalescent;
   and in that, since said heteromannan comprises at least one glucomannan, said heteromannan is not subjected to any treatment with an alkaline agent, notably to any treatment with calcium hydroxide (Ca(OH)₂) or with sodium carbonate.

The invention relates to a process for preparing an essentially non-starchy food solid, to such a food solid and to the uses thereof.

According to the OECD (Organisation for Economic Co-operation and Development), in 2017, one in every two adults and one in every six children worldwide were overweight or obese. In France, the proportion of obese people reached 17% in 2017. Although measures are taken to combat excess weight and obesity in certain countries, the proportion of overweight or obese people is constantly increasing and the OECD forecasts for the coming years follow this increasing trend.

Excess weight and obesity constitute a major risk factor for the occurrence of chronic diseases such as cardiovascular diseases and diabetes, in particular “type-2 diabetes” or “non-insulin-dependent diabetes” (NIDD). Excess weight and obesity are mainly due to an imbalanced high-calorie diet combined with a lack of physical activity and possibly genetic and/or psychological factors predisposing towards excess weight and/or obesity. A diet rich in starchy products (such as bread, pasta and starches in general) and/or sugary products (rich in sucrose) leading to an increase in the blood glucose level, to activation of fatty acid synthesis and of the storage of triglycerides and to inhibition of lipolysis is representative of such an imbalanced high-calorie diet. Such a high-calorie diet is imbalanced in the sense that, as a result of the substantial variations in glycaemia which it brings about, it does not afford a long-lasting sensation of satiety, and as such the consumer feels hungry and feels the need to eat frequently in order to obtain long-lasting satisfaction of his appetite.

The invention is directed towards proposing a food solid in replacement for high-calorie foods.

The invention is directed towards proposing such a replacement food solid which does not lead to a massive supply of glucose in the body during its digestion.

In addition, the excess blood glucose may lead to the phenomenon known as protein “glycation”, which is of a nature to modify the properties of certain proteins, this modification potentially being the cause of cell dysfunctions, pathologies and/or inflammatory and/or autoimmune reactions. Such pathologies are liable to appear all the more frequently in the case of the elderly, presenting with chronic hyperglycaemia (prediabetic or diabetic) and slowed catabolismfor the degradation of “glycation” products.

The invention is thus directed towards proposing a food solid which is capable of limiting protein “glycation”.

Also, a diet which is restricted to products that are starchy and poor in resistant starch and/or sugary, and rapidly hydrolysed during digestion does not make it possible to provide an amount of delayed-hydrolysis soluble fibre necessary for harmonious digestive transit, but also necessary for maintaining and developing the microbiota formed from the intestinal flora, notably from the microbiological flora of the colon.

The consumption of such products that are essentially starchy and/or sugary and poor in soluble and insoluble fibre does not make it possible to afford a sufficient satiety effect that is sufficiently long-lasting and of a nature to prevent the early occurrence of the sensation of hunger.

The invention is directed towards proposing a process for preparing a food solid and such a food solid as a replacement for starchy foods.

Such solutions generally proceed via a consumption of foods that are rich in soluble fibre, non-hydrolysable or sparingly hydrolysable in the stomach and the small intestine, but hydrolysable by microorganisms of the colonic microbiota.

In Japan, “konnyaku” is known, which is traditionally by the calcium hydroxide treatment of a flour of Amorphophallus konjac tubers (“konjac” flour) at a concentration of between 1 and 3 g/L (0.1 to 0.3% by mass) in water. “Konnyaku” is generally used in the form of vermicelli (“shirataki”), in the form of “rice grains” or in slab form. “Konnyaku” is a cohesive solid food. “Shirataki” vermicelli are non-swelling.

U.S. Pat. No. 5,173,321 discloses flavoured “konnyaku” and a process for preparing such a flavoured “konnyaku” in which refined “konnyaku” powder and flavourings are mixed in an amount of water at a temperature of between 20° C. and 30° C. The mass proportion of refined “konnyaku” powder in the mixture is between 2.3% and 4.5%. Gelling of the mixture is obtained subsequently by adding calcium hydroxide. Such a “konnyaku” is non-swelling and does not make it possible to absorb a flavoured liquid medium. It therefore does not allow extemporaneous flavouring by simple contact with a flavoured liquid foodstuff. It does not participate in highlighting the organoleptic properties of a foodstuff. This type of food is also known from EP3406143, WO2008/111195, CN108294259, KR20180057422, CN107691965, WO98/33395 and EP0310703.

However, “konnyaku” and “shirataki” do not increase in volume during digestion and do not afford a sensation of satiety.

Moreover, certain forms of “konnyaku” are packaged in sachets in reaction brine and as such an Occidental consumer is confronted, on opening the sachet, with a strong and unusual odour which he associates with spoiled food. It is essential for this consumer to rinse the “konnyaku” several times.

The invention is directed towards overcoming the drawbacks of these forms of “konnyaku”.

The invention is directed towards proposing a process for preparing a food solid and a food solid having the property of swelling during its gastric transit and which is capable of affording the consumer a sensation of satiety, notably by the effect of filling the stomach.

The invention is directed in particular towards proposing such a food solid which has at least one of the following advantages:
it is low-calorie, notably calorie-free,
it does not contribute towards glycaemia,
it contributes towards supplying soluble fibre favourable to the development of a diversified flora of the intestinal microbiota.

The invention is directed towards proposing a “glucose-free” food solid which is capable of avoiding the recourse to pharmaceutical hypoglycaemiant agents and, where appropriate, to insulin.

The invention is directed towards proposing such a food solid which is rich in dietary fibre. In particular, the invention is directed towards proposing such a food solid, the consumption of which is able to satisfy the recommended daily ration of dietary fibre.

The invention is directed towards proposing such a food solid for its use by a patient suffering from a metabolic disease for which a diet low in sugars and rich in dietary fibre is prescribed.

The invention is directed towards proposing such a food solid which has a balanced ratio between soluble fibre and insoluble fibre.

The invention is directed towards proposing such a food solid for its regulating effect on intestinal transit.

The invention is directed towards proposing such a food solid for its preventive and/or curative use in leaky gut syndrome.

The invention is also directed towards proposing such a food solid rich in soluble fibre, the hydrolysis of which by the microorganisms of the microbiota is slow enough to be able to take place throughout the residence time of said soluble fibre in the colon.

The invention is also directed towards proposing such a food solid in replacement for foods that are rich in fibre but also rich in starch, such as whole cereals, and/or foods that are rich in fibre but also rich in sugars, such as fruits.

The invention is also directed towards proposing such a food solid in replacement for foods that are, indeed, rich in fibre, but the digestibility of which poses a problem, such as certain legumes and certain bulb plants, for example Jerusalem artichoke.

The invention is directed towards proposing such a food solid which has a low glycaemic index.

The invention is directed towards proposing such a food solid for controlling the body mass of its consumer.

The invention is directed towards proposing such a food solid for its use as an agent for slowing down ageing.

The invention is directed towards proposing such a food solid which can absorb at least a portion of sauce of a foodstuff in sauce.

The invention is directed towards proposing such a food solid which is appetizing.

The invention is directed towards proposing a process for preparing a food solid, in which is prepared a mixture:
of an amount of a flour comprising at least one polysaccharide, named heteromannan, chosen from the group formed from glucomannans and galactomannans, and
of an amount of an aqueous liquid composition, characterized in that said heteromannan is in an amount such that the ratio of the mass of said heteromannan in the mixture to the mass of said aqueous liquid composition in the mixture is between 5% and 35%, and
   in that the mixture is prepared by vigorous stirring, via which a substantially homogeneous dispersion is formed, named pourable dispersion, of the flour in the aqueous liquid composition, said pourable dispersion comprising said heteromannan, and having a dynamic viscosity of less than 100 Pa·s, said pourable dispersion then spontaneously changing to form an aqueous cohesive solid which is substantially free of free aqueous liquid composition and which has a dynamic viscosity greater than the dynamic viscosity of said pourable dispersion, and then
   a step of maturation of the aqueous cohesive solid and of hardening of the aqueous cohesive solid is performed so as to form the food solid, the formed food solid being non-adhesive, notably non-adhesive to the touch, and non-coalescent by contact at the temperature of use and atmospheric pressure;
   and in that, since said heteromannan comprises at least one glucomannan, notably at least one glucomannan from Amorphophallus konjac tuber, said heteromannan is not subjected to any treatment with an alkaline agent, notably to any treatment with calcium hydroxide (Ca(OH)₂) or with sodium carbonate.

Throughout the text, the term “flour” commonly denotes a powder with a mean particle size of less than 1 mm, notably less than 500 μm, preferably between 50 μm and 400 μm, more preferentially between 100 μm and 300 μm.

Throughout the text hereinbelow, the term “substantially homogeneous” means that since said pourable dispersion is formed from a homogeneous matrix formed from particles of said flour dispersed in said aqueous liquid composition, the matrix is liable to include lumps of particles of said flour having a proportion of aqueous liquid composition less than the proportion of aqueous liquid composition of said matrix; the ratio of the mass of such lumps to the mass of said pourable dispersion is less than 10%, notably less than 5%, preferably less than 1%. In particular, this ratio is less than 4%, notably less than 3%, preferably less than 2%, more preferentially less than 0.5%. Even more preferentially, this mixing is performed so that said pourable dispersion is free of lumps of particles of said flour.

Throughout the text, the “non-coalescence” denotes the property of the food solid according to the invention via which parts, particles, pieces or fragments formed from this food solid do not fuse spontaneously by simple contact at the temperature of use of the food solid and atmospheric pressure. The term “temperature of use” means the temperature of the foodstuff in which the food solid is intended to be used.

In a process according to the invention, the amount of aqueous liquid composition placed in contact with the amount of flour is less than or equal to the maximum amount of said aqueous liquid composition that is able to be fully absorbed by said amount of flour.

In a process according to the invention, rapid, notably instantaneous, dispersion of the flour in the aqueous liquid composition is performed, via which is formed said pourable dispersion in which the flour particles are distributed in dissociated form in the aqueous liquid composition, the flour particles of said aqueous dispersion changing spontaneously by absorption of the total amount of aqueous liquid composition available to form an aqueous cohesive solid with a dynamic viscosity greater than the dynamic viscosity of said pourable dispersion.

In a process according to the invention, the vigorous stirring is stopped when said pourable dispersion with a dynamic viscosity of less than 100 Pa·s is obtained, and said pourable dispersion is left to change spontaneously to form the aqueous cohesive solid substantially having a dynamic viscosity greater than the dynamic viscosity of said pourable dispersion.

The dynamic viscosity of said pourable dispersion and/or of the aqueous cohesive solid is measured by any means known to those skilled in the art, notably by means of a rotational viscometer or a vibrational viscometer.

The inventor assumes that the step of mixing by vigorous stirring of the flour and of the aqueous liquid composition makes it possible to give a homogeneous dispersion of the flour particles in the aqueous liquid composition, prior to hydration of the particles. This hydration, which is necessarily performed with a restricted amount of aqueous liquid composition, would enable reorganization of the hydrophobic and hydrophilic domains of these particles in dispersed form, leading to the formation of the aqueous cohesive solid. He assumes that the formation of this aqueous cohesive solid proceeds from a constrained reorganization of at least some hydrophobic domains and of at least some hydrophilic domains of said heteromannan, probably from a reorientation of hydrophobic domains and of hydrophilic domains of the particles of said heteromannan, leading to a metastable state of the aqueous cohesive solid, of increased cohesion relative to the cohesion of said pourable dispersion and liable to change by maturation, notably by thermal maturation, towards the stabilized, flexible, non-adhesive, non-coalescent food solid which is capable of being formed into shape by extrusion, notably by hot extrusion. He assumes that this rapid and vigorous dispersion followed by hydration of the flour particles leads to the formation of particles, notably of particles of said heteromannan, in which the hydrophilic domains become reorganized, due to this dispersion and of this subsequent hydration, towards the interior of the particles and the hydrophobic domains become reorganized towards the exterior of the particles, developing cohesive properties.

The inventor observed that the maturation step leads to a change in the appearance of the aqueous cohesive solid, going from an opaque appearance to a translucent appearance. The inventor estimates that this change in appearance, which comes in addition to the abovementioned modifications of the physical properties, reflects the disappearance of the hydrated flour particles in favour of larger-sized objects.

In certain embodiments of a process according to the invention, during the maturation step, the aqueous cohesive solid is left to stand for a period of more than a few hours, notably more than 48 hours, and at room temperature.

However, there is nothing to prevent, in other embodiments, the maturation step from being performed by heating of the aqueous cohesive solid to a temperature above 80° C., notably between 80° C. and 120° C., so as to form the food solid. The maturation step is performed by heating the aqueous cohesive solid for a time sufficient to form the food solid. The inventor assumes that the maturation step enables stabilization of the aqueous cohesive solid and hardening thereof via hydrophobic interactions established between the particles of said flour, notably between the particles of said heteromannan.

In certain embodiments of a process according to the invention, a step of forming into shape of the food solid is performed. This step of forming into shape is performed by extrusion. In certain particular embodiments of a process according to the invention, this step of forming into shape is performed by hot extrusion of the food solid, notably at a temperature of between 80° C. and 120° C. Advantageously, the hot extrusion of the hot food solid is performed on conclusion of the heating step. However, there is nothing to prevent these two steps from being dissociated and from performing this hot extrusion in a heating extruder, starting with a food solid at room temperature. A stable, non-adhesive and non-coalescent extruded food solid is formed.

Via a process according to the invention, the aqueous cohesive solid which is flexible, i.e. deformable, which shows substantially no tensile or compressive elasticity and which is non-adhesive is first formed. By maturation of the aqueous cohesive solid, a food solid which is hardened, cohesive, i.e. a food solid which is stable under the effect of the stresses internal to the food solid, non-adhesive and non-pourable is formed. Such a food solid which is cohesive and which, entirely surprisingly as regards a food solid comprising at least one heteromannan and water, is not adhesive, notably substantially non-adhesive to the touch, is formed.

In certain embodiments of a process according to the invention, said at least one heteromannan is a glucomannan. It may be a swelling glucomannan, which is capable of becoming hydrated, of absorbing the aqueous liquid composition and of increasing its mass by swelling.

Advantageously and according to the invention, mixing is performed by vigorous stirring of said amount of flour and of said amount of aqueous liquid composition, said stirring being maintained for a time of not more than 3 minutes, notably for a time of not more than 2 minutes, preferably for a time of the order of 1 minute.

In a particular embodiment of a process according to the invention, the aqueous liquid composition is at a temperature below +15° C. during the mixing by vigorous stirring. Mixing by vigorous, rapid and continuous stirring of the amount of flour and of the amount of aqueous liquid composition is performed, the aqueous liquid composition being maintained at a temperature below +15° C. In this particular embodiment, the aqueous liquid composition is at a temperature of between +1° C. and +10° C., preferably of the order of +4° C. In this particular embodiment of mixing at low temperature, the ratio of the mass of said heteromannan to the mass of said aqueous liquid composition may be between 15% to 35%. However, there is nothing to prevent the ratio of the mass of said heteromannan to the mass of said aqueous liquid composition from being between 5% and 15%.

Entirely surprisingly and unexpectedly, the inventor has observed that this cold mixing leads to an aqueous cohesive solid, of increased dynamic viscosity relative to the viscosity of said pourable dispersion, which is substantially non-adhesive, notably non-adhesive to the touch, and to a hardened, cohesive, non-pourable, non-adhesive, notably non-adhesive to the touch, food solid which is capable of being extruded to form a non-coalescent and substantially inelastic extruded food solid. Purely for comparative purposes, mixing performed slowly and with insufficiently vigorous stirring does not make it possible to ensure dispersion of the particles before they are hydrated. A highly heterogeneous and light-opaque mixture is formed, containing highly hydrated particles and sparingly hydrated particles. This mixture is very sparingly cohesive and fractures into small-sized fragments, notably by extrusion, and does not enable extrusion as continuous filaments.

In certain advantageous embodiments, the step of maturation of the aqueous cohesive solid and of hardening of the aqueous cohesive solid is performed by heating. In certain advantageous embodiments, the step of maturation of the aqueous cohesive solid and of hardening of the aqueous cohesive solid is performed by heating the aqueous cohesive solid in a closed reactor under the autogenous pressure. In certain embodiments, the aqueous cohesive solid is heated in a closed autoclave at a temperature of the order of 118° C. and for a time sufficient to enable hardening of the aqueous cohesive solid. In certain embodiments, the aqueous cohesive solid is heated under pressure for a time of less than 3 minutes. The food solid formed has greater hardness than the hardness of the aqueous cohesive solid. It is stable, cohesive, substantially non-adhesive to the touch, non-coalescent and capable of being extruded. However, there is nothing to prevent the step of maturation at room temperature and at atmospheric pressure from being performed for a time of several hours or of several days.

In certain embodiments, said heteromannan is in a mass amount such that the ratio of the mass amount of said heteromannan to the mass amount of said aqueous liquid composition is between 5% and 30%, notably between 5% and 25%, in particular between 7% and 23%, preferably between 10% and 20%.

In certain embodiments, the amounts of flour and of aqueous liquid composition in the mixture being mass amounts, the ratio of the amount of flour to the amount of said aqueous liquid composition is between 5% and 60%, notably between 5% and 45%. In certain embodiments, the ratio of the amount of flour to the amount of said aqueous liquid composition is between 5% and 25%, notably between 10% and 20%. In these embodiments, the food solids formed are advantageously intended for the preparation of cold foodstuffs. Such food solids are non-coalescent at their temperature of use. In other embodiments, the ratio of the amount of flour to the amount of said aqueous liquid composition is between 20% and 60%, notably between 20% and 40%. In these other embodiments, the food solids formed are advantageously intended for the preparation of hot foodstuffs, such as hot soups. Such food solids are non-coalescent at their temperature of use, i.e. when hot. The amount of aqueous liquid composition and the amount of flour are adapted according to the desired organoleptic properties.

In certain advantageous embodiments of a process according to the invention, the flour comprises a flour of the Amorphophallus konjac tuber. In certain advantageous embodiments according to the invention, the flour is formed from a flour of the Amorphophallus konjac tuber. In these embodiments, said heteromannan is a glucomannan which has not undergone any treatment with an alkaline agent, notably with calcium hydroxide (Ca(OH)₂) or sodium carbonate. Said heteromannan is an at least partially acetylated glucomannan, which has the property of becoming hydrated and whose mass increases by mixing with an aqueous liquid composition. In these embodiments, the aqueous cohesive solid is substantially translucent.

In certain embodiments of a process according to the invention, alone or in combination, said at least one heteromannan is a galactomannan. It may be a swelling galactomannan, which is capable of becoming hydrated, of absorbing the aqueous liquid composition and of increasing its mass by swelling. In these embodiments, said galactomannan is in a mass amount such that the ratio of the mass amount of said heteromannan to the mass amount of said aqueous liquid composition is between 7% and 35%, notably between 7% and 30%, in particular between 10% and 30%, preferably between 10% and 20%.

In certain embodiments of a process according to the invention, the flour comprises an amount of at least one insoluble fibre. The term “insoluble fibre” means, as is common, a polymer that is not digestible by the digestive enzymes, notably by the amylases, produced in the human digestive tube and not digestible by the constituent symbiotic microorganisms of the digestive and/or intestinal microbiota. Such undigested insoluble fibre does not contribute towards glycaemia and has low calorific power. In these embodiments of a process according to the invention, at least one insoluble fibre is chosen from the group notably formed from celluloses, hemicelluloses, chitins, wheat bran, oat bran, lignins and tannins. It is also possible to use mineral particles such as hydroxyapatite or clay. Products based on “konnyaku” may also be milled and serve as insoluble fibre, as may polymers that have been chemically modified by crosslinking. In certain advantageous embodiments according to the invention, at least one insoluble fibre is cellulose, notably microcrystalline cellulose. In these embodiments of a process according to the invention, the amount(s) of insoluble fibre being mass amounts, the ratio of this (these) amount(s) to the amount of said heteromannan is between 25% and 75%.

In certain embodiments, there is nothing to prevent the flour from comprising an amount of at least one soluble fibre other than said heteromannan. The term “soluble fibre” means, as is common, an oligomer or a polymer that is not digestible by the digestive enzymes, notably by the amylases, produced in the human digestive tube, but which is digestible by the constituent symbiotic microorganisms of the digestive and/or intestinal microbiota. Such soluble fibre, which is digested in the terminal part of the digestive tube (colon), does not contribute towards glycaemia and has low calorific power. It may be oligosaccharides, notably milk oligosaccharides, which are not digestible in the small intestine. It may be a disaccharide, such as lactose for people who are lactose-intolerant. It may be oligosaccharides produced by bacteria, notably by Escherichia coli, which are recombinant or obtained by chemical modification of an oligosaccharide. It may be a resistant starch that is naturally present in many starchy products or that is prepared by partial enzymatic hydrolysis of starch. It may be a yeast fermentation medium or milk fermented by “kefir” or by thermophilic lactic acid bacteria. It may be bacterial exopolysaccharides, glycated proteins or condensed collagen. In certain embodiments of a process according to the invention, said at least one soluble fibre is chosen from the group formed from inulins, pectins, carrageenans and alginates. In these embodiments, the flour comprises a mass amount of at least one soluble fibre such that the ratio of this amount to the amount of said heteromannan is between 25% and 75%.

In certain embodiments of a process according to the invention, the flour has a mean particle size of less than 500 μm, notably between 100 μm and 300 μm.

In certain embodiments of a process according to the invention, the aqueous liquid composition is water. It may be pure water. It may also be a saline buffer that is suitable for adjusting the pH and/or the salinity of said aqueous liquid composition. In any case, the aqueous liquid composition and the food solid are free of any alkaline agent, notably of calcium hydroxide (Ca(OH)₂) and/or of sodium carbonate, for acetate group release of the glucomannan. According to certain embodiments of a process according to the invention, there is nothing to prevent the addition of at least one protein, a fermentation must, notably a fermentation must comprising proteins, at least one food yeast, notably Saccharomyces cerevisiae, or at least one dietetic yeast to said aqueous liquid composition. According to certain embodiments of a process according to the invention, there is equally nothing to prevent the addition of an amount of cellulose to said aqueous liquid composition or the use, as aqueous liquid composition, of any aqueous cellulose preparation obtained from an industrial process. An energy-consuming step of dehydration of the aqueous cellulose preparation is thus avoided.

In certain advantageous embodiments of a process according to the invention, the food solid is subjected to a step of forming into shape by extrusion, notably by hot extrusion, in particular by hot extrusion under pressure. Advantageously, the extrusion of the food solid is performed through a die for forming food pastas (“spaghettis”) into shape. There is nothing to prevent the food solid from being extruded so as to form a noodle form, a rice form, a lasagna form or a “wrap” form. Advantageously, said extrusion is performed in a twin-screw extruder. However, there is nothing to prevent the extrusion from being performed using a household kitchen tool such as a meat mincer.

In certain advantageous embodiments of a process according to the invention, the step of forming into shape by extrusion is a step of coextrusion of a food solid via which an outer surface layer consisting of a first food solid and a subjacent core consisting of a second food solid different from the first food solid are simultaneously formed. In certain advantageous embodiments of a process according to the invention, the first food solid is a food solid rich in “konjac” glucomannans and the second food solid is a food solid rich in guar galactomannans. In certain advantageous embodiments of a process according to the invention, the first food solid is formed from “konjac” flour and the second food solid is a food solid formed from guar flour.

In certain advantageous embodiments, the process according to the invention comprises a step of sterilizing the food solid. Such a sterilization step is performed via any means suitable for the preparation of a sterile food solid that is able to be conserved while substantially preserving its initial organoleptic properties. Such a step of hot sterilization of a food solid placed in a closed container that is hermetic to microorganisms and to fluids, notably to gases, is performed.

In certain advantageous embodiments, the food solid is prepared with a high ratio of the amount of flour to the amount of aqueous liquid composition so that the food solid placed in an aqueous liquid foodstuff becomes hydrated and reaches a chosen degree of dryness to ideally adjust the organoleptic properties of the foodstuff, in particular within a reasonable time period.

The invention also relates to a food solid obtained via a process according to the invention.

The invention relates to a food solid comprising, as a mixture:
an amount of a flour comprising at least one polysaccharide, named heteromannan, chosen from the group formed from glucomannans and galactomannans, and
an amount of an aqueous liquid composition,
   characterized in that said heteromannan is in an amount such that the ratio of the mass of said heteromannan in the food solid to the mass of said aqueous liquid composition in the food solid is between 5% and 35%, and in that the food solid is:
cohesive,
non-adhesive, notably non-adhesive to the touch,
capable of absorbing an amount of an aqueous liquid by placing said food solid and the aqueous liquid in contact, and
suitable for being formed into shape by extrusion;
   and in that, since at least one glucomannan is a glucomannan from Amorphophallus konjac tuber, the food solid is free of any alkaline agent, notably of calcium hydroxide (Ca(OH)₂) and/or of sodium carbonate, for conversion of the native Amorphophallus konjac tuber glucomannan into at least partly deacetylated glucomannan.

The amount of aqueous liquid absorbed by the food solid is evaluated by weighing by immersing an initial amount of food solid into a volume of aqueous liquid at a temperature of 20° C. After the immersion, all of the hydrated food solid is taken up, drained and weighed. The weight gain of the food solid and the uptake of water by the food solid are evaluated.

Advantageously and according to the invention, the food solid is substantially inelastic. The food solid has organoleptic properties of a non-rubbery solid.

Advantageously and according to the invention, the food solid is non-adhesive and non-coalescent by contact of parts of said food solid at the temperature of use and atmospheric pressure.

In certain embodiments, the food solid is suitable for being formed into shape by extrusion.

In certain embodiments, a food solid according to the invention comprises, notably predominantly, at least one glucomannan, notably at least one glucomannan from “konjac” flour, and/or at least one galactomannan, notably at least one guar galactomannan.

In certain embodiments, a food solid according to the invention comprises at least one insoluble fibre, notably cellulose.

In certain embodiments, at least one glucomannan is a glucomannan from Amorphophallus konjac tuber, and the food solid is free of any alkaline agent, notably of calcium hydroxide (Ca(OH)₂) and/or of sodium carbonate, for conversion of the native Amorphophallus konjac tuber glucomannan into at least partly deacetylated glucomannan.

In certain embodiments, the food solid is in the form of filaments that are not coalescent by simple contact with each other, in particular at the temperature of use and atmospheric pressure. The food solid is in fractionated form, each fraction being elongated and having substantially the same shape along any cross section (“spaghettis”), the fractions not being coalescent with each other by contact.

In certain embodiments, the food solid according to the invention is sterile.

In certain embodiments, the food solid according to the invention is packaged in sterile form in a closed container that is hermetic to microorganisms and to fluids, notably to gases. The extruded food solid is non-coalescent, in particular during its hot wet sterilization under pressure.

The food solid according to the invention is free of any alkaline agent, notably of calcium hydroxide (Ca(OH)₂) and/or of sodium carbonate.

The invention also relates to such a food solid for its use as a medicament. The invention also relates to such a food solid for its use as a functional food. The invention also relates to the use of such a food solid in human food and/or animal feed, notably in pet food.

The invention relates to such a food solid for its use for a preventive or curative treatment of at least one of the following pathologies: caries, periodontitis, acne, excess weight, obesity, hypertension, sleep apnoea, impotence, diabetes, arthrosis, neuropathy, microangiopathy, macroangiopathy, infarction, stroke, autoimmune diseases, allergies, neurodegenerative diseases, depression, autism, colopathy, constipation, cancer, psoriasis.

The invention relates in particular to such a food solid for its use as a medicament in a preventive or curative treatment of at least one pathology chosen from metabolic diseases, obesity, diabetes, arterial hypertension, cardiovascular diseases, autoimmune diseases, allergies, neurodegenerative diseases and cancers.

The invention also relates to such a food solid for its use as a medicament in the treatment of digestive and metabolic diseases. The invention also relates to such a food solid for its use as a medicament in the treatment of diabetes and/or of excess weight and/or of digestive disorders.

The invention also relates to such a food solid for its use in food, notably in human food and/or animal feed. The invention also relates to such a food solid for its use as a food supplement and/or as a food replacement for a starchy food.

In certain embodiments, a food solid according to the invention is used in a step for making a foodstuff intended to be consumed cold, notably at a temperature below 25° C. The invention also relates to a foodstuff to be consumed cold and comprising a food solid according to the invention. It may be a “smoothie” comprising a food solid according to the invention to be consumed at low temperature, notably between 1° C. and 10° C. The food solid is not coalescent at its temperature of use of between 1° C. and 10° C. It may be a food solid according to the invention to be consumed in a salad at a temperature of the order of 15° C. to 30° C. The food solid is not coalescent at its temperature of use of between 15° C. and 30° C.

In other embodiments, a food solid according to the invention is used in a step for making a foodstuff intended to be consumed hot, notably at a temperature above 25° C. The invention also relates to a foodstuff to be consumed hot and comprising a food solid according to the invention. It may be a food solid according to the invention to be consumed as a hot meal at a temperature of between 40° C. and 60° C. The food solid is not coalescent at its temperature of use of between 40° C. and 60° C. It may be a food solid according to the invention to be consumed in a hot soup at a temperature of between 60° C. and 100° C. The food solid is not coalescent at its temperature of use of between 60° C. and 100° C.

In certain embodiments of a process according to the invention, a food solid having an outer surface layer consisting of a first food solid and a subjacent core consisting of a second food solid different from the first food solid is prepared. In certain embodiments, a food solid having an outer surface layer consisting of a first food solid and a subjacent core consisting of a second food solid different from the first food solid is prepared by coextrusion of the first food solid of outer surface layer and of the second food solid of subjacent core, in two coaxial directions. In certain embodiments, the first outer food solid consists essentially of at least one glucomannan. In certain embodiments, the first outer food solid consists essentially of at least one glucomannan, notably of “konjac” flour, and the second subjacent food solid consists essentially of at least one galactomannan, notably of guar flour. A food solid which is non-coalescent, in particular non-coalescent during its hot sterilization, is thus formed. Such a food solid is prepared via any means, notably by coextrusion in two coaxial directions of the first subjacent food solid and of the second outer food solid. There is nothing to prevent a food solid according to the invention from being prepared in the form of particles having an internal subjacent core inscribed inside an outer surface layer and enveloped thereby. There is nothing either to prevent the food solid from having at least one layer of at least one additional food solid interposed between the subjacent core, notably the inner core, and the outer surface layer.

A food solid according to the invention has properties of absorbing an aqueous liquid, notably water, and of swelling which is compatible with its use in culinary preparations. In particular, such slow and delayed swelling of a food solid according to the invention during digestion makes it possible to ensure a sensation of satiety for the consumer, limiting the food ration to the amount of food required for his metabolism. By means of its adjusted and balanced composition of insoluble fibre and of soluble fibre, in particular of soluble fibre of the “heteromannan” type and of reduced glycaemic index, it enables rheological regulation of the digestive transit, notably of the intestinal transit, limits the massive supply of glucose during digestion and promotes the development of a varied intestinal microbial flora of the microbiota, which is protective against digestive metabolic pathologies.

The invention also relates to a process for preparing a food solid, to such a food solid, to such a food solid for its use as a medicament and to the uses of such a food solid according to the invention, characterized, in combination or otherwise, by all or some of the characteristics mentioned hereinabove or hereinbelow. Irrespective of the formal presentation that is given thereof, unless explicitly indicated otherwise, the various characteristics mentioned hereinabove or hereinbelow should not be considered as being strictly or inextricably linked together, the invention being able to concern only one of these structural or functional characteristics, or only a portion of these structural or functional characteristics, or only a portion of one of these structural or functional characteristics, or else any group, combination or juxtaposition of all or a portion of these structural or functional characteristics.

Other aims, characteristics and advantages of the invention will emerge on reading the following description and the examples given without any implied limitation, of some of the possible embodiments of the invention.

In a process according to the invention, a food solid is prepared from a flour mainly containing polysaccharides chosen from glucomannans and galactomannans and an aqueous liquid composition via a process according to the invention without forming a highly adhesive solid that is unsuitable for forming into shape by extrusion. A non-adhesive food solid is formed from flour containing “konjac” flour without requiring alkaline treatment of the mixture of the flour and water with calcium hydroxide, which is necessary for obtaining “konnyaku”. The food solid according to the invention does not give off an odour characteristic of “konnyaku” that is unpleasant to an Occidental consumer. The dietary, culinary and gastronomic advantage of the food solid according to the invention arises from its adjustable organoleptic properties. In addition, the food solid according to the invention has properties of swelling in water, which are virtually inexistent in “konnyaku” which is indefinitely stable and does not swell, giving it satiety power that is greater than the satiety power of “konnyaku”. Also, the food solid according to the invention is formed from soluble fibre that is digestible by the microorganisms of the microbiota and to their benefit, in contrast with “konnyaku”, the polysaccharides of which modified by the alkaline treatment are sparingly digestible.

In a process for preparing a food solid according to the invention, a flour comprising at least one polysaccharide chosen from the group formed from glucomannans and galactomannans is chosen or manufactured.

Glucomannans are high molecular weight polysaccharides formed from a main chain consisting of D-glucose and D-mannose linked via β-(1-4) bonds in an irregular distribution of the D-glucose and of the D-mannose. The ratio of the amount of D-mannose and of the amount of D-glucose of a glucomannan, the amounts being expressed in moles, is of the order of 1.6. Certain D-mannose hydroxyls of “konjac” glucomannans bear acetyl groups in position C2 or C3 or C6. Among the glucomannans, glucomannans from Amorphophallus konjac tubers are preferably used.

Galactomannans are polysaccharides formed from a main chain consisting of galactose and mannose linked via β-(1-4) bonds. Among the galactomannans, use is made of at least one galactomannan chosen from the group formed of a galactomannan from fenugreek gum in which the ratio of the number of moles of mannose to the number of moles of galactose is of the order of 1/1, of a galactomannan from guar gum in which the ratio of the number of moles of mannose to the number of moles of galactose is of the order of 2/1, of a galactomannan from tara gum in which the ratio of the number of moles of mannose to the number of moles of galactose is of the order of 3/1, of a galactomannan from locust bean gum in which the ratio of the number of moles of mannose to the number of moles of galactose is of the order of 4/1 or of a galactomannan from cassia gum in which the ratio of the number of moles of mannose to the number of moles of galactose is of the order of 5/1.

In a process for preparing a food solid according to the invention, a flour comprising at least one guar galactomannan is chosen or manufactured. Such a galactomannan enables a supply of galactose which ensures the diversification of the constituent microorganisms of the microbiota.

In certain embodiments, the flour is a mixture of a cellulose powder, notably of microcrystalline cellulose, and of “konjac” flour. In certain embodiments, the cellulose has a mean particle size of between 100 μm and 300 μm. There is nothing to prevent the flour from comprising at least one compound chosen from the group formed from an inulin, lignin, tannins, hemicellulose, collagen, pectin, etc. . .

In a process for preparing a food solid according to the invention, said pourable dispersion is formed by mixing, notably at low temperature and under mechanical stress, of a flour comprising at least one polysaccharide chosen from the group formed from glucomannans and galactomannans in an aqueous liquid composition. This mixing step may be performed by rapidly supplying by sprinkling the amount of flour with vigorous stirring in the amount of cold aqueous liquid composition, notably water. This dispersion is performed so as to transiently form a pourable dispersion with a dynamic viscosity of less than 100 Pa·s which spontaneously changes to form the aqueous cohesive solid substantially free of free aqueous liquid composition and with a dynamic viscosity higher than the dynamic viscosity of said pourable dispersion, and the aqueous cohesive solid is then matured and hardened while hot so as to form the food solid.

After the step of maturation by heating, doughs are extruded at high temperature and under high pressure through dies of an extruder to give products of various shapes and sizes, noodles, rices, lasagnas, wraps, etc.

A food solid according to the invention makes it possible to limit the proportion of carbohydrates (monosaccharides and disaccharides) in the food ration. It also enables long-lasting release of nutrients during digestion. It affords a sensation of satiety by supplying fibre and by swelling of this fibre and avoids the temptation to overconsume foods.

A food solid according to the invention constitutes a non-starchy food that is capable of affording chosen texture and taste organoleptic sensations and satiety by means of a balanced supply of soluble fibre and of insoluble fibre. Such a food solid makes it possible to take into account the digestive specificities of each consumer, such as the age, the gender, the level of physical activity, the rate of digestive transit, the general state of health and the biodiversity and dynamism of the microbiota.

Example 1: “Konjac” flour. A food solid according to the invention is prepared by rapid addition with vigorous stirring of a mass of Amorphophallus konjac tuber flour with a particle size of 120 mesh (Kalys Gastronomie, France) in a mass of water at a temperature of 4° C. Typically, for a volume of 100 mL of water at 4° C., the addition of the corresponding amount of flour is performed in less than 5 seconds, notably about 3 seconds. The amounts of flour and of water are given in Table 1 below. A pourable dispersion which changes into a non-adhesive aqueous cohesive solid is formed. A step of maturation of the non-adhesive aqueous cohesive solid is performed at a temperature of 118° C. under the autogenous pressure in an autoclave or in a pressure cooker. A non-adhesive cohesive food solid of increased hardness relative to the aqueous cohesive solid is obtained. This hardened food solid is subjected to extrusion so as to give the food solid the form of “spaghettis”. Preferably, the food solid having a (mass of flour/mass of water) ratio of greater than or equal to 15% is extruded at high temperature. Preferably, the food solid having a (mass of flour/mass of water) ratio of less than 15% is extruded at low temperature. Preferably, the food solid having a (mass of flour/mass of water) ratio of the order of 5% is extruded at a temperature of the order of +4° C. A non-adhesive, cohesive food solid in the form of “spaghettis” which are substantially non-coalescent by contact is obtained.

The rate of water uptake of the food solid according to the invention is evaluated. An initial amount of food solid is placed in an excess volume of water at a temperature of 20° C. 30, 60, 120 and 180 minutes after mixing the amount of food solid and the amount of water, all of the food solid is taken up, drained and weighed. The weight gain of the food solid and the rate of water uptake by the food solid are thus evaluated. The results are given in Table 1 below, in which column “A” represents the mass (in grams) of flour supplied for the formation of the food solid, column “B” represents the mass (in grams) of water supplied for the formation of the food solid, column “C” represents the ratio of the mass (in grams) of flour supplied for the formation of the food solid to the mass (in grams) of food solid, column “D” represents the increase in mass (in grams) of the food solid maintained for 30 minutes in an excess of water at 20° C., column “E” represents the increase in mass (in grams) of the food solid maintained for 60 minutes in an excess of water at 20° C., column “F” represents the increase in mass (in grams) of the food solid maintained for 120 minutes in an excess of water at 20° C. and column “G” represents the increase in mass (in grams) of the food solid maintained for 180 minutes in an excess of water at 20° C. The values in parentheses represent the ratio (as a percentage) between the variation in mass and the starting mass of the food solid.

TABLE 1

| A | B | | D | E | F | G |
|---|---|---|---|---|---|---|
| Flour, | Water, | | 30 | 60 | 120 | 180 |
| g | g | C | min | min | min | min |
| 5 | 100 | 4.8% | 12 g | 23 g | / | / |
| | | | (11.4%) | (21.9%) | | |
| 7.5 | 100 | 7.0% | 14 g | 27 g | / | / |
| | | | (13.0%) | (25.1%) | | |
| 10 | 100 | 9.1% | 16 g | 33 g | / | / |
| | | | (14.5%) | (30.0%) | | |
| 15 | 100 | 13.0% | 45 g | 80 g | / | / |
| | | | (39.1%) | (69.5%) | | |
| 25 | 100 | 20.0% | 55 g | 98 g | 150 g | 207 g |
| | | | (44.0%) | (78.4%) | (120.0%) | (165.6%) |
| 40 | 100 | 28.5% | 100 g | 180 g | 205 g | 320 g |
| | | | (71.4%) | (128.6%) | (146.4%) | (228.6%) |

An increase in the mass of the food solid immersed in pure water at 20° C. is observed, said increase being indicative of the swelling properties of the food solid according to the invention. The kinetics of increase of the mass of the food solid immersed in an excess of water at a temperature of 20° C. are, however, slow, representative of the metastable state of the constituent material of the food solid and compatible with its use in culinary preparations. These slow kinetics of water uptake by the food solid according to the invention contrast with the excessively rapid kinetics of hydration of the “konjac” flour not treated by means of a process according to the invention. The non-adhesive and hardness properties of the food solid according to the invention contrast with the adhesive properties of the “konjac” flour hydrated by addition of water at room temperature. These non-adhesive and hardness properties of the food solid according to the invention allow forming into shape of the food solid, for example by extrusion, and sterilization thereof at high temperature.

A similar test is also performed by placing an amount of food solid according to the invention in an excess volume of water at a temperature of 100° C. The results are given in Table 2 below, in which column “A” represents the mass (in grams) of flour supplied for the formation of the food solid, column “B” represents the mass (in grams) of water supplied for the formation of the food solid, column “C” represents the ratio of the mass (in grams) of flour supplied for the formation of the food solid to the mass (in grams) of food solid, column “H” represents the increase in mass (in grams) of the food solid maintained for 30 minutes in an excess of water at 100° C. and, in parentheses, the ratio (as a percentage) between the variation in mass and the starting mass of the food solid.

TABLE 2

| A | B | | H | |
|---|---|---|---|---|
| Flour, g | Water, g | C | 15 min | |
| 10 | 100 | 9.1% | 30 g | (28.6%) |
| 15 | 100 | 13.0% | 80 g | (74.4%) |
| 25 | 100 | 20.0% | 128 g | (116.4%) |
| 40 | 100 | 28.5% | 213 g | (185.2%) |

The kinetics of increase of the mass of the food solid immersed in an excess of water at a temperature of 100° C. are, however, slow, representative of the metastable state of the constituent material of the food solid and compatible with its use in culinary preparations.

The food solids described in Example 1 are stable during their subsequent sterilization. From a value of 20% of the ratio of the mass (in grams) of flour supplied for the formation of the food solid to the mass (in grams) of food solid and for values of this ratio of greater than 20%, no coalescence of the “spaghettis” is observed during their hot sterilization. In particular, the “spaghettis” dissociate by simple immersion in water. However, there is nothing to prevent the food solid from being prepared extemporaneously, notably in the form of “spaghettis”, for the purpose of using it in a culinary preparation.

The food solids described in Example 1 formed from Amorphophallus konjac tuber flour essentially contain glucomannans. Since the upper part of the human digestive tube, formed by the mouth, the pharynx, the oesophagus, the stomach and the small intestine, is not equipped with an enzymatic composition suitable for the digestion of glucomannans, the food solids described in Example 1 reach the colon without having been hydrolysed, without having released glucose, do not significantly contribute towards glycaemia and have a calorific power of the order of 2 kcal per gram of glucomannan. They are hydrolysed by the microorganisms of the colon which produce enzymes suitable for this hydrolysis, releasing glucose and mannose that may be used by the microorganisms of the colon for their own metabolism and maintenance of their diversity.

It should be noted that refined starchy foods such as white bread and white rice are hydrolysed and reabsorbed before reaching the small intestine and do not contribute towards maintaining and developing microorganisms of the microbiota. Exclusive consumption of such refined starchy foods results in the decline of at least a portion of these microorganisms and impoverishment of the microbiota.

Example 2: “Guar” flour. A food solid according to the invention is prepared via a process as described in Example 1, by rapid addition with vigorous stirring of a mass of guar flour (Kalys Gastronomie, France) in a mass of water at a temperature of 4° C. Typically, for a volume of 100 mL of water at 4° C., the addition of the corresponding amount of flour is performed in less than 5 seconds, notably about 3 seconds. The amounts of flour and of water are given in Table 3 below, in which column “I” represents the mass (in grams) of “guar” flour supplied for the formation of the food solid, column “J” represents the mass (in grams) of water supplied for the formation of the food solid, column “K” represents the ratio of the mass (in grams) of flour supplied for the formation of the food solid to the mass (in grams) of food solid. The food solid formed from guar flour according to a process in accordance with the process described in Example 1 is cohesive and non-adhesive and is of increased hardness relative to the food solid formed on conclusion of the mixing at 4° C. This hardened food solid is subjected to extrusion so as to give the food solid the form of “spaghettis”. The rate of water uptake of the extruded food solid according to the invention is evaluated as described in Example 1. In Table 3, columns “L”, “M” and “N” represent the increase in the mass (in grams) of the food solid maintained for 60 minutes, 120 minutes and 210 minutes, respectively, in an excess of water at 20° C. and, in parentheses, the ratio (as a percentage) between the variation in mass and the starting mass of the food solid.

TABLE 3

| I | J | | L | M | N |
|---|---|---|---|---|---|
| Flour, g | Water, g | K | 60 min | 120 min | 210 min |
| 10 | 100 | 9.1% | 35 g | / | / |
| | | | (31.8%) | | |
| 15 | 100 | 13.0% | 80 g | / | / |
| | | | (69.6%) | | |
| 25 | 100 | 20.0% | 108 g | 160 g | 172.8 g |
| | | | (86.4%) | (128.0%) | (120.0%) |
| 40 | 100 | 28.6% | 134 g | 217 g | 311 g |
| | | | (95.7%) | (155.0%) | (222.1%) |

An increase in the mass of the food solid immersed in pure water at 20° C. is observed, said increase being indicative of the swelling properties of the food solid according to the invention. The kinetics of increase of the mass of the food solid immersed in an excess of water at a temperature of 20° C. are, however, slow, representative of the metastable state of the constituent material of the food solid and compatible with its use in culinary preparations.

The food solids described in Example 2 are stable during their subsequent sterilization. From a value of 25% of the ratio of the mass (in grams) of flour supplied for the formation of the food solid to the mass (in grams) of food solid and for values of this ratio of greater than 25%, no coalescence of the “spaghettis” is observed during their hot sterilization and their storage in hot sterilized form.

However, there is nothing to prevent the food solid, notably in the form of “spaghettis”, from being prepared extemporaneously and without sterilization for the purpose of using it in a culinary preparation. A device of the “food ricer/press” type commonly used in cookery is advantageously used for cold extrusion. There is nothing either to prevent the food solid from being stored at low temperature, notably at a freezing temperature, for the purpose of its subsequent use in a culinary preparation.

Example 3: “Konjac” and “guar” mixed flour. A food solid according to the invention is prepared via a process as described in Example 1, by rapid addition with vigorous stirring of a mass of a mixture of Amorphophallus konjac “konjac” tuber flour (Kalys Gastronomie, France) and guar flour (Kalys Gastronomie, France) in a mass of water at a temperature of 4° C. Typically, for a volume of 100 ml of water at 4° C., the addition of the corresponding amount of flour is performed in less than 5 seconds, notably about 3 seconds. The amounts of flour and of water are given in Table 4 below, in which column “N” represents the mass (in grams) of “konjac” flour supplied for the formation of the food solid, column “O” represents the mass (in grams) of “guar” flour supplied for the formation of the food solid, column “P” represents the mass (in grams) of water supplied for the formation of the food solid, column “Q” represents the ratio of the mass (in grams) of flour supplied for the formation of the food solid to the mass (in grams) of food solid. The food solid formed from the “konjac” and “guar” mixed flour according to a process in accordance with the process described in Example 1 is cohesive and non-adhesive and is of increased hardness relative to the food solid formed on conclusion of the mixing at 4° C. This hardened food solid is subjected to extrusion so as to give the food solid the form of “spaghettis” by means of a food ricer/press.

The rate of water uptake of the extruded food solid according to the invention is evaluated as described in Example 1. In Table 4, columns “R” and “S” describe the increase in the mass (in grams) of the food solid maintained for 60 minutes and 120 minutes, respectively, in an excess of water at 20° C. and, in parentheses, the ratio (as a percentage) between the variation in mass and the starting mass of the food solid.

TABLE 4

| N | O | | | | |
|---|---|---|---|---|---|
| “Konjac” | “Guar” | P | | R | S |
| flour, g | flour, g | Water, g | Q | 60 min | 120 min |
| 20 | 20 | 100 | 28.6% | 177 g | 233 g |
| | | | | (126.4%) | (166.4%) |

An increase in the mass of the food solid immersed in pure water at 20° C. is observed, said increase being indicative of the swelling properties of the food solid according to the invention. The kinetics of increase of the mass of the food solid immersed in an excess of water at a temperature of 20° C. are, however, slow, representative of the metastable state of the constituent material of the food solid and compatible with its use in culinary preparations.

Example 4: Mixed flour of “konjac” and of insoluble fibre, cellulose. A food solid according to the invention is prepared via a process as described in Example 1, by rapid addition with vigorous stirring of a mass of a mixture of Amorphophallus konjac “konjac” tuber flour (Kalys Gastronomie, France) and cellulose in a mass of water at a temperature of 4° C. Typically, for a volume of 100 ml of water at 4° C., the addition of the corresponding amount of flour is performed in less than 5 seconds, notably about 3 seconds. The amounts of flour and of water are given in Table 5 below, in which column “T” represents the mass (in grams) of “konjac” flour supplied in the flour for the formation of the food solid, column “U” represents the mass (in grams) of microcrystalline cellulose supplied in the flour for the formation of the food solid, column “V” represents the mass (in grams) of water supplied for the formation of the food solid, column “W” represents the ratio of the mass (in grams) of flour supplied for the formation of the food solid to the mass (in grams) of food solid.

The food solid formed from the “konjac” and cellulose mixed flour according to a process in accordance with the process described in Example 1 is cohesive and non-adhesive and is of increased hardness relative to the food solid formed on conclusion of the mixing at 4° C. This hardened food solid is subjected to extrusion so as to give the food solid the form of “spaghettis” by means of a food ricer/press.

The rate of water uptake of the extruded food solid according to the invention is evaluated as described in Example 1. In Table 5, columns “X” and “Y” describe the increase in the mass (in grams) of the food solid maintained for 60 minutes and 120 minutes, respectively, in an excess of water at 20° C. and, in parentheses, the ratio (as a percentage) between the variation in mass and the starting mass of the food solid.

TABLE 5

| T | | | | | |
|---|---|---|---|---|---|
| “Konjac” | U | V | | X | Y |
| flour, g | Cellulose, g | Water, g | W | 60 min | 120 min |
| 20 | 20 | 100 | 28.6% | 109 g | 144 g |
| | | | | (77.9%) | (102.8%) |

An increase in the mass of the food solid immersed in pure water at 20° C. is observed, said increase being indicative of the swelling properties of the food solid according to the invention. The kinetics of increase of the mass of the food solid immersed in an excess of water at a temperature of 20° C. are, however, slow, representative of the metastable state of the constituent material of the food solid and compatible with its use in culinary preparations. It was observed that the use of a flour formed by mixing “konjac” flour and microcrystalline cellulose makes it possible to form a food solid which is of increased stability in comparison with the food solid obtained with “konjac” flour alone. It was also observed that the use of “konjac” flour masks the unpleasant taste of the cellulose, notably for a value of the ratio of the mass of “konjac” flour to the mass of cellulose of greater than or equal to 25% and for a proportion of “konjac” flour in the food solid of greater than or equal to 9% (ratio of the mass of “konjac” flour to the mass of food solid of greater than or equal to 10%).

Example 5: Mixed food solid produced from a “konjac” and cellulose mixed flour. A food solid according to the invention is prepared via a process as described in Example 1, in which the flour is a mixed flour comprising mass proportions of 50% of “konjac” flour and 50% of cellulose. 30 g of said mixed flour are rapidly mixed in 100 ml of water at a temperature of +4° C. with vigorous stirring. A cohesive, non-adhesive and substantially non-coalescent food solid in which the unpleasant taste of the cellulose is masked is obtained.

Example 6: Mixed food solid produced from a “konjac” and cellulose mixed flour. A food solid according to the invention is prepared via a process as described in Example 1, but in which the flour is a mixed flour comprising mass proportions of 1/3 of “konjac” flour and 2/3 of cellulose. 30 g of said mixed flour are mixed in 100 ml of water at a temperature of +4° C. A cohesive, non-adhesive and substantially non-coalescent food solid in which the unpleasant taste of the cellulose is masked is obtained.

Example 7: Mixed food solid produced from a “konjac” and cellulose mixed flour. A food solid according to the invention is prepared via a process as described in Example 1, but in which the flour is a mixed flour comprising mass proportions of 50% of “konjac” flour and 50% of cellulose. 40 g of said mixed flour are mixed in 100 ml of water at a temperature of +4° C. A cohesive, non-adhesive and substantially non-coalescent food solid in which the unpleasant taste of the cellulose is masked is obtained.

Example 8: Mixed food solid produced from a “konjac” and cellulose mixed flour. A food solid according to the invention is prepared via a process as described in Example 1, but in which the flour is a mixed flour comprising mass proportions of 25% of “konjac” flour and 75% of cellulose. 40 g of said mixed flour are mixed in 100 ml of water at a temperature of +4° C. A cohesive, non-adhesive and substantially non-coalescent food solid in which the unpleasant taste of the cellulose is masked is obtained.

Example 9: Mixed food solid produced from a “konjac”, cellulose, inulin, tara and pectin mixed flour. A food solid according to the invention is prepared via a process as described in Example 1, but in which the flour is a mixed flour comprising about 33% of “konjac” flour, about 33% of cellulose, about 11% of inulin powder, about 11% of tara powder and about 11% of pectin powder. 45 g of said mixed flour are mixed in 100 ml of water at a temperature of +4° C. A cohesive, non-adhesive and substantially non-coalescent food solid in which the unpleasant taste of the cellulose is masked is obtained.

Example 10: Mixed food solid produced from a “konjac” and cellulose mixed flour. A food solid according to the invention is prepared via a process as described in Example 1, but in which the flour is a mixed flour comprising 50% of “konjac” flour and 50% of cellulose. 30 g of said mixed flour are mixed in a suspension comprising 15 g of dietetic yeast in 100 ml of water at a temperature of +4° C. A cohesive, non-adhesive and substantially non-coalescent food solid in which the unsuitable and unpleasant tastes of the cellulose and of the dietetic yeast are masked is obtained. There is nothing to prevent such a food solid from being used for masking the taste of any other ingredient or condiment.

The extruded food solids described in Examples 1 to 10 have organoleptic properties that are compatible with their use in a culinary preparation. The food solids according to the invention are rich in heteromannans, notably in glucomannans and/or galactomannans, and make it possible to reduce the glycaemic impact of these food solids relative to the glycaemic impact of essentially starchy compositions.

A food solid according to the invention is balanced in soluble fibre and insoluble fibre. It is shear-thinning and allows rheologically controlled digestive transit. It forms, by means of digestion, a viscous, adhesive and hydrophilic material suitable for the development and maintenance of the microorganisms of the microbiota.

Example 11: Preparation of a foodstuff. A food solid according to the invention is prepared via a process as described in Example 1, in which the flour is a “konjac” flour. By extrusion of the aqueous cohesive solid through a die, a cohesive food solid is obtained, in the form of filaments or “spaghettis”, which is non-adhesive and substantially non-coalescent. The extruded food solid is mixed with a tomato-based sauce. The inventor observed diffusion of the tomato sauce into the filaments of the food solid, forming a peripheral envelope which opposes the coalescence.

The invention may be the subject of numerous variants and applications other than those described hereinabove. In particular, it goes without saying that, unless otherwise indicated, the various structural and functional characteristics of each of the embodiments described hereinabove must not be considered as combined and/or strictly and/or inextricably linked to each other, but, on the contrary, as simple juxtapositions. In addition, the structural and/or functional characteristics of the various embodiments described hereinabove may form the subject totally or partly of any different juxtaposition or of any different combination.

Other applications may be envisaged in the food sector. They may be used as replacement for fat, gelatin, entrails, unfermentable soluble fibre in snack products or as a stabilizer and water activity-limiting agent. Other applications may be envisaged in the field of food films and in the cosmetics field for modification of the water activity.

1. Process for preparing a food solid, in which is prepared a mixture:
of an amount of a flour comprising at least one polysaccharide, named heteromannan, chosen from the group formed from glucomannans and galactomannans, and
of an amount of an aqueous liquid composition,
characterized in that said heteromannan is in an amount such that the ratio of the mass of said heteromannan in the mixture to the mass of said aqueous liquid composition in the mixture is between 5% and 35%, and
in that the mixture is prepared by vigorous stirring, via which a substantially homogeneous dispersion is formed, named pourable dispersion, of the flour in the aqueous liquid composition, and having a dynamic viscosity of less than 100 Pa·s, said pourable dispersion then spontaneously changing to form an aqueous cohesive solid which is substantially free of free aqueous liquid composition and which has a dynamic viscosity greater than the dynamic viscosity of said pourable dispersion, and then
a step of maturation of the aqueous cohesive solid and of hardening of the aqueous cohesive solid is performed so as to form the food solid, the formed food solid being non-adhesive and non-coalescent by contact at the temperature of use and atmospheric pressure;
and in that, since said heteromannan comprises at least one glucomannan, said heteromannan is not subjected to any treatment with an alkaline agent, notably to any treatment with calcium hydroxide (Ca(OH)₂) or with sodium carbonate.

2. Process according to claim 1, characterized in that the aqueous liquid composition is at a temperature below +15° C. during the mixing by vigorous stirring.

3. Process according to either of claims 1 and 2, characterized in that the flour comprises an Amorphophallus konjac tuber flour.

4. Process according to one of claims 1 to 3, characterized in that the flour comprises an amount of at least one insoluble fibre.

5. Process according to claim 4, characterized in that the flour comprises cellulose as insoluble fibre.

6. Process according to one of claims 1 to 5, characterized in that the flour has a mean particle size of less than 500 μm.

7. Process according to one of claims 1 to 6, characterized in that the aqueous liquid composition is water.

8. Process according to one of claims 1 to 7, characterized in that the food solid is subjected to a step of forming into shape by extrusion through a die for forming food spaghettis.

9. Process according to one of claims 1 to 8, characterized in that a food solid having an outer surface layer consisting of a first food solid and a subjacent core consisting of a second food solid different from the first food solid is prepared by coextrusion of the first food solid of outer surface layer and of the second food solid of subjacent core, in two coaxial directions.

10. Process according to one of claims 1 to 9, characterized in that it comprises a step of sterilization of the food solid.

11. Food solid comprising, as a mixture:
an amount of a flour comprising at least one polysaccharide, named heteromannan, chosen from the group formed from glucomannans and galactomannans, and
an amount of an aqueous liquid composition,
characterized in that said heteromannan is in an amount such that the ratio of the mass of said heteromannan in the food solid to the mass of said aqueous liquid composition in the food solid is between 5% and 35%, and in that the food solid is:
cohesive,
non-adhesive,
capable of absorbing an amount of an aqueous liquid by placing said food solid and the aqueous liquid in contact, and
suitable for being formed into shape by extrusion;
and in that, since at least one glucomannan is a glucomannan from Amorphophallus konjac tuber, the food solid is free of any alkaline agent for conversion of a native Amorphophallus konjac tuber glucomannan into at least partly deacetylated glucomannan.

12. Food solid according to claim 11, characterized in that it is in the form of filaments that are not coalescent by simple contact with each other at the temperature of use and atmospheric pressure.

13. Food solid according to either of claims 11 and 12, for its use as a medicament.

14. Use of a food solid according to either of claims 11 and 12 in food.
